(19) United States
(12) Patent Application Publication
(10) Pub. No.: US 2008/0243243 A1
(43) Pub. Date: Oct. 2, 2008

(54) Implantable medical articles having pro-healing coatings

(76) Inventors: Stuart K. Williams, Harrods Creek, KY (US); David E. Babcock, St. Louis Park, MN (US); Joseph A. Chinn, Shakopee, MN (US); David L. Clapper, Duluth, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/824,908

(22) Filed: Jul. 3, 2007

Related U.S. Application Data
(60) Provisional application No. 60/819,091, filed on Jul. 7, 2006.
(60) Provisional application No. 60/848,588, filed on Sep. 29, 2006.

(51) Int. Cl. A61F 2/06

(52) U.S. Cl. 623/1.47; 623/1.46; 623/1.44; 623/1.15

(57) ABSTRACT

Coatings including adhesion factors for the surfaces of implantable medical articles are disclosed. The coatings are used to improve the function of the device by promoting a pro-healing response following implantation. The coatings can modulate endothelialization of the article surface to reduce the risk of adverse tissue responses that may reduce the functionality of the device.

### CROSS-REFERENCE TO RELATED APPLICATIONS

The present non-provisional Application claims the benefit of commonly owned provisional Application having Ser. No. 60/819,091, filed on Jul. 7, 2006, and entitled IMPLANTABLE MEDICAL ARTICLES HAVING PRO-HEALING COATINGS; and commonly owned provisional Application having Ser. No. 60/848,588, filed on Sep. 29, 2006, and entitled IMPLANTABLE MEDICAL ARTICLES HAVING PRO-HEALING COATINGS; which Applications are incorporated herein by reference in their entirety.

### FIELD OF THE INVENTION

The invention relates to coatings for implantable medical article and methods for promoting a pro-healing response.

### BACKGROUND OF THE INVENTION

Until more recently, the primary focus of advances in implantable medical article technology has been to alter a structural characteristic of the article to improve its function within the body. However, it has become appreciated that function of the implanted device at the site of implantation can be greatly enhanced by improving the compatibility of the devices in the context of the tissue response that occurs as a result of the implantation. Ideally, improved compatibility would allow surfaces of the implanted device to mimic natural tissue exposed by an injury and provide an environment for the formation of normal tissue as a result of the healing process.

Despite being inert and nontoxic, implanted biomaterials associated with the device, such as various plastics and metals, often trigger responses such as inflammation, fibrosis, infection, and thrombosis. If excessive, some of these reactions may cause the device to fail in vivo. A moderate cellular inflammatory response is commonly seen immediately following implantation, wherein leukocytes, activated macrophages, and foreign body giant cells are recruited to the surface of the implanted device. While the inflammatory response is common and generally a component of the healing process, it often culminates in the formation of a substantial fibrous matrix on the surface of the implanted device.

Many aspects of tissue responses to the vascular or coronary placement of metal stents have been studied are understood. Generally, there are at least three phases of the vascular response to the implantation of metal stents. These phases include attachment of coagulation factors, cell recruitment leading to inflammation, and cellular proliferation (see, for example, Edelman E. R. and Rogers, C. (1998) Am. J. Cardiol., 81:4E-6E). The extent of these responses is typically dictated by the extent of the tissue damage in the area of stent deployment.

The sequence of events following placement of a metallic stent generally begins with attachment of coagulation factors. In this stage a thin protienaceous membrane forms, covering the vascular and stent surface. Coagulation factor deposition is most commonly observed within 1-3 days of stent implantation. The proteinaceous membrane is formed by the adhesion of factors such as fibrinogen (and subsequently fibrin) and von Willebrand factor (vWF) on the stent surface, which form a loosely structured matrix. This phase is also characterized by platelet adhesion.

The coagulation factors that attach to the stent surface in the initial phase function as the endoluminal layer of the vessel wall in the first weeks after stenting. The extent of coagulation factor attachment can be affected by the presence of systemic anticoagulants, which are commonly administered in association with a stenting procedure.

Inflammatory and cell recruitment generally follow the stage associated with the attachment of coagulation factors. Following thrombosis, an increased number of inflammatory cells, such as leukocytes and macrophages, are found associated with the thrombotic layer. This period occurs about 3-7 days after stent implantation. During this period, changes in the adhesion of inflammatory cells are also seen, with a decrease in adhering leukocytes and an increase in macrophages that are thought to form multinucleated giant cells around the stents.

This stage is also associated with the presence of endothelial cells (ECs) and smooth muscle cell (SMCs) on the stent surface. The attachment of endothelial cells and formation of an endothelial cell layer on an implant can modulate the thrombotic and inflammatory response occurring on the surface of the stent. This is thought to be beneficial, as the risk of forming occlusions near the stent surface is reduced.

Formation of an endothelial cell layer on the surface is also thought to be beneficial from a healing standpoint. Normal tissue responses in the vicinity of the stent are promoted and undesirable tissue responses that could compromise function of the stent are minimized. Ideally, a mature endothelium is formed in association with the stent surface following a period of implantation. Mature endothelial cells can modulate other cellular responses, such as the proliferation of SMCs.

While the attachment and formation of an endothelial cell layer is desirable, it is also associated with a proliferative phase. It is thought that cell proliferation results in a substantial increase in ECs and/or SMCs in association with the stent surface. While moderate proliferation of ECs is desirable, excessive proliferation of ECs may also be associated with hyperproliferation of SMCs. Hyperproliferation of SMCs can lead to hyperplasia and restenosis. Given this, it is thought that promoting a moderate EC response on the stent surface is a way of forming a mature endothelial cell layer, promoting a natural healing response, and limiting the hyperproliferation of SMCs that is commonly associated with traditional stenting procedures.

Also, more recently, tissue responses to the vascular or coronary placement of metal stents provided with a drug eluting coating have become better understood.

Generally, placement of drug-eluting stents is accompanied by a prolonged systemic anticoagulation therapy (typically greater than six months) to promote endothelialization of the device surface. Even in the case that this therapy is performed, endothelialization of the device surface is suboptimal.

### SUMMARY

The present invention generally relates to implantable medical articles having coatings that improve the function of the article in vivo. The invention also relates to methods for using these coated medical articles in a subject. Generally, the coated medical articles promote one or more physiological events associated with a pro-healing response. The medical articles of the present invention include a coating having at least one adhesion factor (e.g., matrix proteins, active portions thereof, or binding members thereof) formed on the surface of the device in a manner that provides a particularly desirable endothelial cell response, which can occur on the blood contacting surface of the device.

In one aspect, the coatings of the invention are formed on a body member of an implantable device and include an adhesion factor, a photogroup and a polymeric material. The polymeric material is present in a layer between the surface of the body member and the adhesion factor. In forming the coating, the photogroup is activated to bond the adhesion factor to the polymeric material, or to crosslink the adhesion factor on the surface of the device. The adhesion factor can be a matrix protein such as a collagen or a laminin. In some particular aspects the collagen is collagen I. In some aspects the photogroup chemistry is used to form a coating with collagen in non-fibrillar form. The coatings of the invention can be formed on the surface of stents, many of which are commonly formed of metal or metal alloy material.

In vivo studies associated with the invention show that coatings that include a adhesion factor immobilized using photogroup chemistry provide particularly desirable levels of endothelialization following a period of implantation. In other words, the coatings promote attachment of endothelial cells, but do so in a manner that also results in limiting the proliferation of other cell types on the surface. This can be important, particularly for medical devices, such as stents, that are implanted for a substantial period of time for the treatment of a medical condition.

The coatings of the present invention can be formed on coronary stents to provide a pro-healing response. This pro-healing response is characterized by a modulated formation of an endothelial cell layer that also can limit the proliferation of smooth muscle cells. This in turn can reduce the incidence of restenosis an improve stent function and lifetime.

In some aspects of the invention, the photogroup and adhesion factor are used in conjunction with polymeric material that forms a coated layer and a bioactive agent that is elutable or releasable from the coated layer. The adhesion factor, which is immobilized by the photogroup, improves an otherwise sub-optimal or abnormal endothelial cell response, which is observed on devices when the bioactive-releasing layer is used as the coating alone. This aspect of the invention is advantageous as it can improve therapy for devices with drug-eluting coatings, which typically require a prolonged systemic anticoagulation therapy.

In some aspects, the invention provides a coated intravascular medical device comprising a body member having a body member surface, and a bioactive agent-releasing coating on the body member surface, the coating further comprising an adhesion factor and a photogroup. The bioactive agent-releasing coating comprises a first layer that is in contact with tissue or body fluid, wherein the first layer comprises, predominantly, an adhesion factor having a pendent photogroup. The coating also includes a second layer located between the body member surface and the first layer, the second layer comprising a polymeric material and a bioactive agent. The photogroup bonds the adhesion factor to the polymeric material.

In a related aspect, the invention provides a method for improving the endothelialization of a surface of an implantable medical article comprising a bioactive agent coating. The method comprises providing a medical article with a coating comprising a polymeric material, a bioactive agent, an adhesion factor, and a photogroup that bonds the adhesion factor to the polymeric material. Another step in the method includes implanting the coated medical article in a subject, wherein the coating promotes a level of endothelialization in the subject that is greater than a level of endothelialization observed without the adhesion factor and photogroup.

The coatings can be formed on the surface of devices that would otherwise promote an undesirably high level of endothelialization (such as a bare metal surface of a stent). The coating including the photogroup and adhesion factor can also be used to modulate the endothelial response on the surfaces of these types of implantable devices. In some aspects, the coatings of the invention are used to modulate the endothelialization on surfaces that, for example, after a period of implantation, promote hyperproliferation of smooth muscle cells. Therefore, in some aspects, the coatings can provide a positive, lower level of endothelialization beneficial for the function of devices that are implanted in the body for a prolonged period of time.

In particular photo-collagen coated stents showed modulated endothelialization after a period of implantation, showing formation of an endothelial cell monolayer. By comparison, uncoated (bare metal) stents trended towards endothelial cell hypertrophy, observed by higher levels of endothelialization (endothelial cells attaching in an amount greater than a cellular monolayer).

Therefore, in another aspect, the invention provides a coated intravascular medical device that has a body member comprising a metal or metal alloy and having a body member surface, and a coating on the body member surface. The coating includes a first layer that is in contact with tissue or body fluid, and includes, predominantly, an adhesion factor comprising a pendent photogroup; and a second layer located between the body member surface and the first layer, the second layer comprising a polymeric material. The photogroup bonds the adhesion factor to the polymeric material. For example, the polymeric material can be a compliant synthetic polymer such as poly(para-xylylene). The coating can comprise a first coated layer comprising the second component, and a second coated layer comprising the adhesion factor coupled to the second component via a photoreactive group.

In a related aspect, the invention provides a method for modulating the endothelialization of a surface of an implantable medical article. One step in the method comprises obtaining information regarding the endothelialization of a surface of the medical article, wherein the medical article has a first level of endothelialization when implanted into a subject after a period of time, and the first level of endothelialization is associated with an undesirable tissue response. Another step in the method comprises providing a medical article with a coating comprising at least one adhesion factor and a photogroup to form a coated medical article. Another step in the method includes implanting the coated medical article in a subject, wherein the coating promotes a second level of endothelialization in the subject that is less than the first level of endothelialization after the period of time. In some aspects, the undesirable tissue response is smooth muscle cell hyperproliferation. In some aspects, the subject is a human and the period of time is about two weeks, or greater than two weeks. In some aspects the period of time is about four weeks.

In some aspects, the methods comprise providing a coating to an intraluminal prosthesis, an intravascular prosthesis, or a stent. The stent can be selected from the group of stents used to treat a cardiovascular condition.

In some aspects, the step of implanting is performed by delivering the medical article to an intravascular location in the subject. The coated article is then implanted a subject and maintained for a period of time sufficient to cause the formation of an endothelial layer of cells on a surface of the medical article.

In another aspect, the invention provides a coated intravascular medical device comprising a body member formed of a biodegradable polymer, and having a coating comprising an adhesion factor and a photogroup. The photogroup can bond the adhesion factor to the biodegradable polymer of the body member.

### BRIEF DESCRIPTION OF THE DRAWINGS

FIGS. 1a-1d are scanning electron micrograph (SEM) images (75×) of the surfaces of coated and uncoated stents explanted at day 7 from New Zealand white rabbits. FIG. 1a is a bare metal stent (BMS); FIG. 1b is a drug-eluting coated stent (DES); FIG. 1c is a BMS with a HBPR/Laminin-1 coating; FIG. 1d is a DES with a supplemental HBPR/Laminin-1 coating.

FIGS. 2a-2d are immunofluorescence micrograph images of cells stained with BBI (a nuclei stain) on the surfaces of coated and uncoated stents explanted at day 7 from New Zealand white rabbits. FIG. 2a is a BMS; FIG. 2b is a DES; FIG. 2c is a BMS with a HBPR/Laminin-1 coating; FIG. 2d a DES with a supplemental HBPR/Laminin-1 coating.

FIG. 3 is a graph of the endothelial response on the surface of metal stents and metal stents coated with adhesion factors explanted at day 7 from New Zealand white rabbits.

FIG. 4 is a graph of the endothelial response on the surface of metal stents and metal stents coated with adhesion factors explanted at day 14 from New Zealand white rabbits.

FIGS. 5A-5F are scanning electron micrograph (SEM) images (12× and 35×) of the surfaces of coated and uncoated stents explanted at day 7 from New Zealand white rabbits. FIGS. 5A and 5B is a bare metal stent (BMS); FIGS. 5C and 5D is a bare metal stent (BMS) with a collagen I coating; FIGS. 5E and 5F is a bare metal stent (BMS) with a laminin 1 coating.

FIGS. 6A-6F are scanning electron micrograph (SEM) images (12× and 35×) of the surfaces of coated and uncoated stents explanted at day 14 from New Zealand white rabbits. FIGS. 6A and 6B is a bare metal stent (BMS); FIGS. 6C and 6D is a bare metal stent (BMS) with a collagen I coating; FIGS. 6E and 6F is a bare metal stent (BMS) with a laminin 1 coating.

FIGS. 7A-7C are scanning electron micrograph (SEM) images of the surfaces of coated and uncoated stents taken from a porcine ex-vivo AV shunt model, showing thrombotic responses.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The coatings and methods of the invention can be used for promoting the endothelialization of the coated surface of the article. Endothelialization refers to the attachment and formation of a persistent layer of endothelial cells on the surface of an implanted medical device. Endothelialization of a surface can improve function of the device (such as a stent) and can take place within the context of a broader pro-healing response.

Endothelialization can be beneficial by preventing neointimal accumulation, thereby reducing the likelihood of restenosis of the implanted device. The coatings of the invention that promote endothelialization can also decrease the incidence of subacute and late stent thrombosis by providing a nonthrombogenic surface. These coatings can promote rapid adherence of endothelial cells, leading to a well-formed and persistent endothelial cell layer.

In some aspects, the present invention provides devices, coatings, and methods wherein endothelialization occurs in a modulated manner. This means that the coating of the present invention promotes the attachment of endothelial cells to the coated surface and the formation of a mature endothelial cell layer, but limits the highly proliferative response that is sometimes observed on other surfaces that also promote an endothelial cell response. Endothelialization can be beneficial by preventing neointimal accumulation, thereby reducing the likelihood of restenosis of the implanted device.

The coatings that promote endothelialization in a modulated manner can be formed using the adhesion factors described herein. The adhesion factor can include a component selected from the group of factors that binds to a member of the integrin family of proteins. For example, the coating can include a factor selected from collagen, laminin-5, vitronectin, entactin, tenascin, thrombospondin, and ICAM (Intercellular Adhesion Molecule). Active portions of these adhesion factors can also be used, as well as binding members to these factors.

In some aspects, the coating can include an antibody against a cell surface antigen involved in adhesion. For example, the coating can include an antibody against CD34, or a binding member of CD34, such as MadCAM or L-selectin. Anti-CD34 monoclonal antibodies can bind progenitor endothelial cells from human peripheral blood. These progenitor cells are capable of differentiating into endothelial cells. (Asahara et al. (1997) Science 275:964-967.) Hybridomas producing monoclonal antibodies directed against CD34 can be obtained from the American Type Tissue Collection. (Rockville, Md.).

Studies shown herein demonstrate the endothelialization of a stent surface in a double injury-iliac artery rabbit model using the inventive coatings. Coatings including adhesion factors were formed on both bare metal stents and stents having a previously formed drug-eluting coating. The stents were implanted into rabbits and removed after 7 days and 14 days and endothelial cell adhesion was evaluated on the stents.

The present coatings were able to promote rapid endothelialization of the stent surface. Notably, the endothelial layer was well formed and persisted after its formation (i.e., cell adherence was not transient). These desirable characteristics are supported by observations showing insignificant or no evidence of fibrin deposits on the surface of the coated stents. In comparison, fibrin deposits were observed on stents having only a drug-eluting coating.

These characteristics of the endothelialized surfaces were rather remarkable, given the coated stents were placed in vivo and therefore exposed to a variety of naturally occurring cells and components, including immune cells, thrombogenic components, as well as endothelial cells. The desirable endothelialization of stents that included collagen in the coating was also surprising in view of some collagen coatings of the prior art which have been shown to rapidly attract platelets, leading to a highly thrombogenic surface.

Studies shown herein also demonstrate a therapeutically acceptable level thrombosis of a stent surface in an ex-vivo porcine AV shunt model using photogroup-immobilized collagen. The surface of the photogroup-immobilized collagen showed a desirable low level of thrombosis compared to a collagen coating not formed with photogroup chemistry, which showed excessive, undesirable thrombosis.

The coatings including the adhesion factor and photogroup (without a drug-eluting (DE) matrix) were able to promote a modulated endothelialization of the stent surface. This modulated endothelialization provided coverage with endothelial cells at a level that was less than the level observed with stents not having a coating of the present invention. This lower level of endothelial cell coverage can correlate with reduced proliferation of smooth muscle cells. Such a modulated endothelialization is desirable, as it can reduce the rate of undesirable tissue responses that lead to stent failure. Stent failure is typically characterized by smooth muscle cell hyperproliferation and restenosis at the implantation site.

Generally, the coatings of the present invention include an adhesion factor, an active portion thereof, or a binding member thereof, immobilized on the surface of the implantable medical article using a photogroup. According to some aspects of the invention, a collagen-based coating is described. The implantable medical article can be an article that is introduced into a mammal for the prophylaxis or treatment of a medical condition.

Implantable medical articles include, but are not limited to vascular implants and grafts, grafts, surgical devices; synthetic prostheses; vascular prostheses including stents, endoprosthesis, stent-graft (such as abdominal aortic aneurysms (AAA) stent-grafts), and endovascular-stent combinations; small diameter grafts, abdominal aortic aneurysm grafts; wound dressings and wound management devices; hemostatic barriers; mesh and hernia plugs; patches, including uterine bleeding patches, atrial septal defect (ASD) patches, patent foramen ovale (PFO) patches, ventricular septal defect (VSD) patches, pericardial patches, epicardial patches, and other generic cardiac patches; pericardial sacks; ASD, PFO, and VSD closure devices; percutaneous closure devices, mitral valve repair devices; heart valves, venous valves, aortic filters; venous filters; left atrial appendage filters; valve annuloplasty devices; implantable electrical leads, including pacemaker and implantable cardioverter defibrillator (ICD) leads; catheters; neuro aneurysm patches; central venous access catheters, vascular access catheters, abscess drainage catheters, drug infusion catheters, parental feeding catheters, intravenous catheters (e.g., treated with antithrombotic agents), stroke therapy catheters, blood pressure and stent graft catheters; anastomosis devices and anastomotic closures; aneurysm exclusion devices, such as neuro aneurysm coils; biosensors including glucose sensors; birth control devices; cosmetic implants including breast implants, lip implants, chin and cheek implants; cardiac sensors; infection control devices; membranes; tissue scaffolds; tissue-related materials including small intestinal submucosal (SIS) matrices; shunts including cerebral spinal fluid (CSF) shunts, glaucoma drain shunts; dental devices and dental implants; ear devices such as ear drainage tubes, tympanostomy vent tubes, and cochlear implants; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices including bladder slings, renal devices and hemodialysis devices, colostomy bag attachment devices; biliary drainage products.

Other exemplary devices include self-expandable septal, patent ductus arteriosus (PDA), and patent foramen ovale (PFO) occluders constructed from nitinol wire mesh and filled or associated with polyester fabric (available from, for example, AGA Medical, Golden Valley, Minn.).

In some aspects of the invention, the coating of the present invention is formed on an intraluminal prosthesis. Examples of intraluminal prosthesis include self-expanding stents, balloon-expanded stents, degradable coronary stents, non-degradable coronary stents, peripheral coronary stents, esophageal stents, ureteral stents, and urethral stents. In many cases the intraluminal prosthesis is an intravascular prosthesis.

While the coatings of the present invention can be formed on any implantable medical device where it is desired to form an endothelial cell layer in a modulated manner, intravascular stents are exemplified. Numerous stent constructions have been described and are well known in the art and can benefit from a coating of the present invention. The present coating can be formed on virtually any stent construction available given the teachings herein and/or teachings that are known in the art.

A medical article having an adhesion factor-containing coating can also be prepared by assembling an article having two or more “parts” (for example, pieces of a medical article that can be put together to form the article) wherein at least one of the parts has a coating. All or a portion of the part of the medical article can have an adhesion factor-based coating. In this regard, the invention also contemplates parts of medical articles (for example, not the fully assembled article) that have a coating of the present invention.

General classes of materials from which the medical article can be formed include natural polymers, synthetic polymers, metals, and ceramics. Combinations of any of these general classes of materials can be used to form the implantable medical article.

Metals that can be used in to form the implantable article (such as a stent) include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35.

The implantable medical article can be formed from synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, dextran, dextran sulfate, polydimethylsiloxanes, and polyetherketone.

In some cases the coating of the invention is formed on an implantable medical article is partially or entirely fabricated from a degradable polymer. The article can degrade in an aqueous environment, such as by simple hydrolysis, or can be enzymatically degraded.

Examples of classes of synthetic polymers that can be used to form the structure of the article include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with the device of the invention include polylactide, polygylcolide, polydioxanone, poly(lactide-co-glycolide), poly(glycolide-co-polydioxanone), polyanhydrides, poly(glycolide-co-trimethylene carbonate), and poly(glycolide-co-caprolactone).

In some aspects, the coating includes a first layer that is in contact with tissue or body fluid comprising an adhesion factor having a pendent photogroup, and a second layer located between the body member surface and the first layer, the second layer comprising a polymeric material. The second coated layer can facilitate formation of the layer that includes the adhesion factor and photogroup.

The second coated layer can be a base layer of polymeric material that is formed on the surface of the implantable article. For example, the first coated layer can be a base coat of polymeric material formed on a metal stent, such as a Parylene™ layer, or a silane-containing layer, such as hydroxy- or chloro-silane.

Parylene™ (poly(para-xylylene) base layers are typically very thin (0.1 micron to 75 microns), continuous, inert, transparent, and conformal films. Parylene™ is applied to substrates in an evacuated deposition chamber by a process known as vapor deposition polymerization (VDP). This involves the spontaneous resublimation of a vapor that has been formed by heating di-para-xylylene, which is a white crystalline powder, at approximately 150° C., in a first reaction zone. The vapor resulting from this preliminary heating is then cleaved molecularly, or pyrolized, in a second zone at 650° C. to 700° C. to form para-xylylene, a very reactive monomer gas. This monomer gas is introduced to the deposition chamber, where it resublimates and polymerizes on substrates at room temperature and forms a transparent film. In the final stage, para-xylylene polymerizes spontaneously onto the surface of objects being coated. The coating grows as a conformal film (poly-para-xylylene) on all exposed substrate surfaces, edges and in crevices, at a predictable rate. Parylene™ formation is spontaneous, and no catalyst is necessary.

A process for forming a Parylene™ base layer on the surface of a metal stent is described in detail in U.S. Publication No. 2005/0244453, filed Nov. 3, 2005 (Stucke et al.).

In a one method, the coating is formed by providing a base layer of Parylene on the article surface, and then attaching a photo-adhesion factor to the base layer via the photo-group. As an example, a metal stent with a Parylene™ coating is provided. A photo-adhesion protein, such as photo-collagen I, is disposed on the Parylene™ coating. The surface of the stent is then treated with UV light, which activates the photogroup, resulting in the bonding of the collagen to the Parylene™ layer.

The process can be carried out by immersing the Parylene™ coated stent in a composition that includes the photo- adhesion protein and then treating the composition with UV light. In many aspects the concentration of photo-adhesion protein is about 5 μg/mL or greater, or about 10 μg/mL or greater. Photogroup-derivatized matrix proteins can be prepared as described in U.S. Pat. No. 5,744,515 (Clapper).

Referring to embodiments wherein the coating comprises a crosslinked layer of polypeptide components, the coating can be formed by providing an adhesion factor, such as collagen, comprising a photoreactive group (i.e., photo-collagen). In these aspects, photo-collagen can be activated to crosslink to other components in the coating composition, including other photo-collagens.

Alternatively, the coating can be formed by combining the components of the coating composition with a coupling moiety that is a photoreactive crosslinking agent. The photoactivatable crosslinking agent can be non-ionic or ionic. The photoactivatable cross-linking agent can include at least two latent photoreactive groups that can become chemically reactive when exposed to an appropriate actinic energy source.

In one mode of practice, the coatings of the invention are used to improve the function of medical articles that include a drug-eluting coating, such as drug-eluting stents. The coatings of the present invention allow one or more adhesion factor(s) to be presented in a manner sufficient to elicit an endothelial cell attachment and the formation of an endothelial cell layer on the surface of the device. In addition, the present methods maintain the drug-releasing properties of the coating, and also the overall desirable physical properties of the coating, such as conformal and compliant properties.

In some incidences drug-releasing stents, such as drug eluting stents, are subject to failure due to adverse tissue responses such as restenosis. In this regard, the adhesion factor coating of the present invention can also be formed in association with stents having a drug releasing coating, and provide an overall benefit for improving stent function in vivo. Examples of stents having drug releasing polymer systems are described in, for example, U.S. Pat. No. 6,669,980, which teaches preparation of medical devices having coatings that include poly(styrene-isobutylene-styrene), U.S. Pat. No. 6,214,901, which teaches coating compositions based on poly(alkyl(meth)acrylate) and poly(ethylene-co-vinyl acetate) mixtures suitable for preparing coatings for hydrophobic drug (such as rapamycin) release, and other hydrophobic polymer systems useful for drug delivery such as described in U.S. Patent Publication Nos. 2005/0220843 and 2005/0244459.

Degradable polymers can also be used as the polymer that includes the bioactive agent that is releasable from the coating. Examples of degradable polymers can include those with hydrolytically unstable linkages in the polymeric backbone. Degradable polymers of the invention include both those with bulk erosion characteristics and those with surface erosion characteristics.

Exemplary synthetic degradable polymers can be selected from the group of polyesters such as poly(lactic acid) (poly(lactide)), poly(glycolic acid) (poly(glycolide)) poly(lactide-co-glycolide), poly(dioxanone); polylactones such as poly(caprolactone) and poly(valerolactone), copolymers such as poly(glycolide-co-polydioxanone), poly(glycolide-co-trimethylene carbonate), and poly(glycolide-co-caprolactone); poly(ether ester) multiblock copolymers such as poly(ethylene glycol) (PEG)/poly(butylene terephthalate) (PBT) block copolymers (see U.S. Pat. No. 5,980,948) and co-polyester consisting glycolide-ε-caprolactone segment and a lactide-glycolide segment; poly(3-hydroxybutyrate), poly(3-hydroxyvalerate), poly(tartronic acid), poly(β-malonic acid), poly(propylene fumarate); degradable polyesteramides; degradable polyanhydrides and polyalkeneanhydrides (such as poly(sebacic acid), poly(1,6-bis(carboxyphenoxy)hexane, poly(1,3-bis(carboxyphenoxy)propane); degradable polycarbonates and aliphatic carbonates; degradable polyiminocarbonates; degradable polyarylates; degradable polyorthoesters; degradable polyurethanes; degradable polyphosphazenes; degradable polyhydroxyalkanoates; degradable polyamides; degradable polypeptides; copolymers thereof, and multi-block copolymers as described in EP1555278.

In some aspects the degradable polymer is a hydrophobic polysaccharide. Exemplary hydrophobic polysaccharides with pendent hydrophobic groups include fatty acid derivatized poly-α(1→4)glucopyranose polymers, such as described in U.S. patent application Ser. No. 11/724,553, filed Mar. 15, 2007 (Chudzik).

In some cases, the drug-eluting coating can include a drug that is sensitive to irradiation of a wavelength that is emitted from a source used to activate the photoreactive groups. For example, the drug may be subject degradation when irradiated with wavelengths in the range of 300 nm or less. Exemplary compounds that may be subject to degradation when irradiated with wavelengths of less than 300 nm include, but are not limited to, sirolimus (rapamycin; Aₘₐₓ=˜290 nm), analogs of rapamycin (“rapalogs”), tacrolimus, ABT-578, everolimus, paclitaxel (Aₘₐₓ=˜231 nm), and taxane.

In order to minimize degradation of the drug in the drug eluting coating, the coated layer including the photogroup can be formed using a filter. Preferably, a filter is used that promotes activation of the photogroup but minimizes degradation of the drug. Typically, filters are identified by the wavelength of light that is permitted to pass through the filter. Exemplary types of filters that can be used in connection with the invention include those selected from ultra-violet cut-off filters, ultra-violet transmitting filters, band pass filters, and colored filters.

In some cases a hydrophilic drug, such as another polypeptide, that is not coupled to the surface of the device can be present in the coated layer that includes an adhesion factor, such as collagen or laminin. In these cases, the hydrophilic drug can be released from the coating while the collagen and/or laminin remains coupled to the surface.

In some aspects, the coating of the present invention includes a collagen, or an active portion thereof. For example, the coating can include a collagen selected from collagen I and collagen IV.

In some aspects, the coating includes a combination of adhesion factors including a collagen adhesion factor and one or more other adhesion factors. In some modes of practice the coating is formed using collagen I or collagen IV, and an adhesion factor that is not a collagen or collagen derived.

Collagen I can be coated on the device to provide fibrillar or non-fibrillar collagen coated surfaces. In many aspects, the coating is formed in a method which provides collagen I in non-fibrillar form.

For example photo-collagen-I can be prepared in a composition having a low pH (e.g., ˜pH 2.0) and used to coat the surface of the implantable article, forming a coating that is non-fibrillar. Raising the pH of the solution (to, e.g., ˜pH 9.0) promotes the self-assemble into fibrils.

A stent having a collagen coating can be formed by a method including the steps of (a) providing a stent, (b) forming a coating on the stent comprising a photoreactive group and a adhesion factor, wherein the step of forming comprises a substep of activating the photoreactive group to immobilize the adhesion factor in the coating.

In some aspects, the stent comprises a metal or metal alloy material. Therefore, a method for forming a collagen coating can include (a) providing a stent comprising a coated layer of polymeric material, (b) forming a coated layer comprising collagen and a photogroup, wherein the photogroup is activated to form a coated layer of collagen on the polymeric material.

In some aspects of the invention the coating includes a laminin, or an active portion thereof. The laminin protein family includes multidomain glycoproteins that are naturally found in the basal lamina. Laminins are heterotrimers of three non-identical chains: one α, β, and γ chain that associate at the carboxy-termini into a coiled-coil structure to form a heterotrimeric molecule stabilized by disulfide linkages. Each laminin chain is a multidomain protein encoded by a distinct gene. Several isoforms of each chain have been described. Different alpha, beta, and gamma chain isoforms combine to give rise to different heterotrimeric laminin isoforms.

The coating on the implantable medical article can include laminin-5 or an active portion thereof. Laminin-5 is composed of the gamma 2 chain along with alpha 3 and beta 3 chains (laminin α3β3γ2) chains. It is synthesized initially as a 460 kD molecule that undergoes specific proteolytic cleavage to a smaller form after being secreted into the ECM. The size reduction is a result of processing the α3 and γ2 subunits from 190-200 to 160 kD and from 155 to 105 kD, respectively. Laminin-5 is an integral part of the anchoring filaments that connect epithelial cells to the underlying basement membrane.

The coating can include an active portion of laminin-5, which may be one or more of the chains of laminin-5, a portion of one of the chains, or combinations thereof. In some aspects, the laminin α3 chain, or a portion thereof, is included in the coating on the implantable medical article. A portion of the laminin α3 chain has a globular structure and is referred to as the G domain, which, it itself, is composed of five tandem repeats referred to as LG repeats. One of the modules within the G domain, referred to as the LG3 module, has been shown to replicate key Ln-5 activities including cell adhesion, spreading, and migration (Shang, M., et al. (2001) J. Biol. Chem. 276:33045-33053. The sequence of the human LG3 modules is available as NCBI (National Center for Biotechnology Information) number A55347.

In one aspect the coating includes a polypeptide having the LG3 sequence of the laminin α3 chain.

Other shorter peptides within the G domain may also be used in the present coatings, such as the peptide sequences PPFLMLLKGSTR and NSFMALYLSKGR.

Laminin-5 can be obtained from various cell lines including HaCaT (spontaneously immortalized human keratinocytes; Boukamp, P., et al. (1988) J. Cell Biol 106:761-771), and HT-1080 (human fibrosarcoma; ATCC, CCL-121). Polyclonal antibodies against laminin-5 are commercially available from, for example, Abcam (#ab14509; Cambridge, Mass.); monoclonal antibodies against laminin-5 chains are commercially available from, for example, Chemicon (mouse anti-laminin-5 γ2 subchain MAb; Temecula, Calif.) and Transduction Laboratories (mouse anti-laminin-5 β3 subchain MAb; Lexington, Ky.), or can be prepared based on a laminin-5 sequence (e.g., rabbit anti-laminin-5 a3 subchain polyclonal (RB-71) as prepared by Bethyl Laboratories, Inc. (Montgomery, Tex.) against the peptide CKANDITDEVLDGLNPIQTD (see Examples)).

Complete nucleic acid and protein sequences are available for the human laminin-5 α3, β3, and γ2 chains. Given this information and the techniques available to one of skill in the art, a desired laminin-5 portion, can be obtained using techniques such as immunopurification, recombinant protein products, or by peptide synthesis.

A coating having laminin-5 activity can also be prepared by providing a coating that includes a component that specifically binds to laminin-5, or a portion thereof, herein referred to as a “binding member.” Antibodies against laminin-5, and portions thereof, are commercially available and described herein. The coating can be prepared by substituting an antibody against laminin-5 for laminin-5 in the coating, or supplementing the coating with an antibody against laminin-5.

In another aspect of the invention, laminin-5, or a portion thereof, is present as the predominant polypeptide in a layer of the coating. That is, laminin-5, or a portion thereof, is present at greater than 50% of the total amount of polypeptide present in the coated layer.

The coating can also include combinations of adhesion factors, such as combinations of collagen or laminin, active portions thereof, or binding members thereof. Another combination includes laminin-1, or an active portion thereof, or a binding member thereof and collagen, or an active portion thereof, or a binding member thereof. Preferred collagens are selected from the group of collagen I and collagen IV.

Photoreactive groups, broadly defined, are groups that respond to specific applied external light energy to undergo active specie generation with resultant covalent bonding to a target. Photoreactive groups are those groups of atoms in a molecule that retain their covalent bonds unchanged under conditions of storage but which, upon activation, form covalent bonds with other molecules. The photoreactive groups generate active species such as free radicals, nitrenes, carbenes, and excited states of ketones upon absorption of external electromagnetic or kinetic (thermal) energy. Photoreactive groups may be chosen to be responsive to various portions of the electromagnetic spectrum, and photoreactive groups that are responsive to ultraviolet, visible or infrared portions of the spectrum are preferred. Photoreactive groups, including those that are described herein, are well known in the art. The present invention contemplates the use of any suitable photoreactive group for formation of the inventive coatings as described herein.

Photoreactive groups, or photoreactive groups that have been activated and that have bonded to a target (e.g., a photoreacted group) are collectively referred to herein as photogroups.

Photoreactive groups can generate active species such as free radicals and particularly nitrenes, carbenes, and excited states of ketones, upon absorption of electromagnetic energy. Photoreactive groups can be chosen to be responsive to various portions of the electromagnetic spectrum. Those that are responsive to the ultraviolet and visible portions of the spectrum are typically used.

Photoreactive aryl ketones such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives can be used. Examples of aryl ketones include heterocyclic derivatives of anthrone, including acridone, xanthone, and thioxanthone, and their ring substituted derivatives. Some photoreactive groups include thioxanthone, and its derivatives, having excitation energies greater than about 360 nm.

These types of photoreactive groups, such as aryl ketones, are readily capable of undergoing the activation/inactivation/reactivation cycle described herein. Benzophenone is a particularly preferred latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benezensulfonyl azide; and phosphoryl azides [(RO)₂PON₃] such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetatoacetates (—CO—CN₂CO—O—) such as t-butyl alpha diazoacetoacetate.

Other photoreactive groups include the diazirines (—CHN₂) such as 3-trifluoromethyl-3-phenyldiazirine; and ketenes (CH═C═O) such as ketene and diphenylketene.

The photogroups can be pendent from an adhesion factor, and the photogroup-derivatized adhesion factor can be used to prepare the coatings of the invention. Photogroup-derivatized matrix proteins can be prepared as described in U.S. Pat. No. 5,744,515 (Clapper).

In some modes of preparation, the photogroup is provided as a crosslinking agent. For example, the adhesion factor-based coating, can be formed using a non-ionic photoactivatable cross-linking agent having the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”).

Ionic photoactivatable cross-linking agents can also be used to form the adhesion factor-based coating. Some ionic photoactivatable cross-linking agents are compounds having the formula: X₁—Y—X₂, wherein Y is a radical containing at least one acidic group, basic group, or a salt of an acidic group or basic group. X₁ and X₂ are each independently a radical containing a latent photoreactive group. For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In some aspects of the invention, the one or more adhesions factors are associated with the surface of the medical article via a hydrophilic polymer. The hydrophilic polymer layer can impart hydrophilic properties to the coating. In some coating arrangements, the second layer includes the hydrophilic polymer and the hydrophilic polymer has pendent first and second reactive groups. In some aspects, the first reactive group comprises a photoreactive group. The second reactive groups can be individually reactive with the adhesion factor. For example, second reactive groups can be amine-reactive groups individually bonding the amine bearing residues of a polypeptide adhesion factor.

In some modes of practice, the first reactive group allows for crosslinking of hydrophilic polymer to form a coated layer. For example, the first reactive group can be activated to react and bond to another hydrophilic polymer, forming a network of hydrophilic polymer as a layer on the surface of the implantable medical article. Such a crosslinked network of hydrophilic polymer may be formed when there is little or no reactivity of the first reactive group and the surface of the article. In some cases, the first reactive group is pendent from the hydrophilic polymer. Preferably, the first reactive group includes a photo-reactive group as described herein.

Alternatively, the network of hydrophilic polymer formed as a layer on the surface of the implantable medical article is formed by the combining a polymeric component with a crosslinking agent, such as crosslinking agent comprising photoreactive groups, as described herein.

In some cases, the hydrophilic polymer is coupled to the surface of the article by the reaction of the first reactive group, such a photoreactive group, with the surface of the article. In this case, the polymeric component can be covalently bonded to the surface of the article.

The second reactive group allows for bonding of the adhesion factor, such as collagen or laminin, and in some cases, one or more other adhesion factors. The second reactive groups are individually reactive with the adhesion factor, such as collagen or laminin, and one or more other adhesion factors. For example, second reactive groups can be amine-reactive groups, such as N-oxysuccinimide (NOS) groups. Other amine-reactive groups include, aldehyde, isothiocyanate, bromoacetyl, chloroacetyl, iodoacetyl, anhydride, isocyanate and maleimide groups.

This arrangement can be used to provide one adhesion factor to the surface, but is particularly advantageous when a combination of two or more adhesion factors, such as collagen and another adhesion factor, are immobilized on the surface. One exemplary combination includes laminin and collagen. Prior to disposing, these polypeptide components (including an adhesion factor such as laminin) can be combined at a desired ratio or concentrations, and then disposed on the polymeric component with reactive second groups. Each polypeptide component can individually react with second reactive groups coupling the polypeptides to the polymer component. In this regard, processing steps are minimized. These improve the efficiency and reduce costs associated with the coating procedure.

The hydrophilic polymer that is used to form the adhesion factor-based coating, such as a laminin-containing coating, can be a synthetic polymer, a natural polymer, or a derivative of a natural polymer. Exemplary natural hydrophilic polymers include carboxymethylcellulose, hydroxymethylcellulose, derivatives of these polymers, and similar natural hydrophilic polymers and derivatives thereof.

In another preferred aspect, the polymer is hydrophilic and synthetic. Synthetic hydrophilic polymers can be prepared from any suitable monomer including acrylic monomers, vinyl monomers, ether monomers, or combinations of any one or more of these types of monomers. Acrylic monomers include, for example, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, and derivatives and/or mixtures of any of these. Vinyl monomers include, for example, vinyl acetate, vinylpyrrolidone, vinyl alcohol, and derivatives of any of these. Ether monomers include, for example, ethylene oxide, propylene oxide, butylene oxide, and derivatives of any of these. Examples of polymers that can be formed from these monomers include poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, for example, methyl vinyl ether/maleic anhydride copolymers and vinyl pyrrolidone/(meth)acrylamide copolymers. Mixtures of homopolymers and/or copolymers can be used.

In exemplary modes of practice the hydrophilic polymer is a (meth)acrylamide copolymer, such as one formed from (meth)acrylamide and (meth)acrylamide derivatives.

Alternatively, a step in the coating process can involve pre-mixing the hydrophilic polymer with one or more adhesion factor(s). This pre-mixture can then be disposed on the surface of an article. For example, a hydrophilic polymer including a first photoreactive group, and a second reactive group that can react with a portion of the adhesion factor, is mixed with the adhesion factor. One, or more than one, adhesion factors can be included in the pre-mixture and can become bonded to the hydrophilic polymer. The pre-mixture is then disposed on the surface the article. The first photoreactive groups can then be activated to bond the hydrophilic polymer/adhesion factor(s) to the surface of the article. In some modes of practice, a polymer base coat (such as Parylene™) is formed on the surface, which the hydrophilic polymer/adhesion factor(s) becomes bonded to.

In yet other aspects, the coating can be formed using an adhesion factor comprising a pendent coupling moiety that is a polymerizable group. The polymerizable group can be an ethylenically unsaturated group. Exemplary ethylenically unsaturated groups include vinyl groups, acrylate groups, methacrylate groups, ethacrylate groups, 2-phenyl acrylate groups, acrylamide groups, methacrylamide groups, itaconate groups, and styrene groups.

In the process of forming the coating, the adhesion factor comprising a pending polymerizable group can be reacted to form a polymerized matrix of adhesion factor, or mixtures of adhesion factors. In some aspects, a collagen macromer is used to form the coating. A collagen macromer suitable for use in forming the present coatings is described in Example 12 of U.S. Pub. No. US-2006/0105012A1. Other adhesion factor macromers, such as laminin macromers, can be prepared using an analogous process.

Formation of the coating including the adhesion factor macromer can be initiated by a polymerization initiator comprising a photogroup. In some cases a photoinitiator is used to promote initiation of a free radical polymerization reaction leading to the formation of a coated layer of polymerized material. Other agents that facilitate formation of a polymerized layer can be present in the composition. These can include, for example, polymerization accelerants which can improve the efficiency of polymerization. Examples of useful accelerants include N-vinyl compounds, particularly N-vinyl pyrrolidone and N-vinyl caprolactam. Such accelerants can be used, for instance, at a concentration of between about 0.01% and about 5%, and preferably between about 0.05% and about 0.5%, by weight, based on the volume of the coating composition. In the course of preparing the coating using the polymeric coating component, it was found that use of the polymeric component to form a coated layer prior to disposing laminin resulted in additional processing and functional advantages.

A modulated endothelial cell response can be measured in various ways. One way of observing this modulation is to histologically compare the surface of an article having a coating of the present invention with that of an article having an uncoated surface or having a chemically different coating. The histological comparison can be carried out after a time of implantation in a mammal. For example, in a test animal such as a rabbit histological examination can be carried out after a period of about 7 and/or a period of about 14 days. In a human subject, this period of time would correlate to about at least about two weeks, on average about four weeks, and in the range of about two weeks to about eight weeks.

Explanted samples can be examined using reagents that allow for the detection of cells associated with the surface of the stents. In some methods of assessment, observation of endothelial cells is performed by treating the explanted stents with BBI (bisbenzimide; Hoechst 33258). Observation of endothelial cells can also be performed by treating the explanted stents with Evans blue dye (Imai, H., et al. (1982) Arch Pathol Lab Med. 106:186-91).

The presence of endothelial cells can also be determined using antibodies to CD31, BS1 lectin, and factor VIII (Krasinski, K., et al. (2001) Circulation 104:1754). Antibodies against these proteins or lectins are commercially available, from, for example Calbiochem (San Diego, Calif.)

In many cases, endothelial cells can be morphologically distinguished from other cell types such as certain immune cells.

Smooth muscle cells can be distinguished from other cell types such as endothelial cells and fibroblasts using antibodies against actin (see, for example, Chamley, J. H., et al. (1977) Cell Tissue Res. 177:445-57).

Scanning electron microscopy can also be carried out to provide higher magnification of the surfaces of explanted stents.

The surfaces of the explanted stents can be scored according to endothelial cell coverage. The density of endothelial cells per unit area of the stent can be performed. In some cases a scoring system can be employed to assess the level of endothelialization. For example at a first level the stent surface has essentially no cells; at a second level the stent surface has some interspersed cells; at a third level the stent surface has localized cell density in certain areas; at a fourth level the stent surface has a consistent cell density covering most of the stent; and at a fifth level the cell density is the highest and cell coverage masks the stent.

As an example, to determine the effectiveness of the coatings of the present invention at modulating an endothelial cell response, information is obtained regarding the level of endothelialization of the stent surface from a stent that does not include the adhesion factor coating of the present invention after a period of implantation in a subject. After a period of implantation, a higher level of endothelialization, such as a level of four, or greater than four, according to the rating system, is determined on average for the uncoated stents. An adhesion factor coating of the present invention is then applied to the uncoated stents and placed in subjects for the determined period of implantation. The adhesion factor coating provides a higher level of endothelialization that is statistically lower than the uncoated stent. For example the level of endothelialization in the coated stents is lower than four.

The invention will be further described with reference to the following non-limiting Examples.

### Testing and Analysis

A heterobifunctional polyacrylamide reagent (HBPR, made as described in Example 9 of U.S. Pat. No. 5,858,653) that contains amine-reactive and photo-reactive groups was used for the preparation of some coatings.

Non-derivatized matrix proteins were obtained from the following sources: bovine collagen-I (Kensey Nash), human collagen-IV (BD Biosciences), human fibronectin (BD Biosciences), mouse laminin-I (BD Biosciences), and human laminin-V (University of Arizona).

### Scanning Electron Microscopy

Samples were prepared for scanning electron microscopy evaluation by dehydration, critical point drying, and sputter coating using a gold target. The samples were evaluated and photomicrographs obtained using a JEOL 820 scanning electron microscope (JEOL USA, Peabody, Mass.).

### Inflammation

Inflammatory response was evaluated using the sections stained with F4/80 viewed under a 40× water-immersion objective lens. Using a 54×54 μm² high power field, 10 fields were randomly selected in the tissue at the tissue-polymer interface, along the entire outer curve of the implant disc. F4/80 positively staining cells within the HPF were counted. Inflammatory response for each implant group was expressed as mean number of F4/80 positive cells/mm²±s.e.m.

### Histology and Immunohistochemistry

Fixed tissue samples were dehydrated, embedded in paraffin, sectioned at 6 μm and processed for histological and immunocytochemical evaluation. General histological structure was determined with hematoxylin and eosin staining. The vasculature was identified using the lectin, GS-1. Samples were evaluated immunocytochemically for the presence of activated macrophages using an antibody against the F4/80 160 kD glycoprotein antigen (biotin-monoclonal, 1:100 Serotec, Inc., Raleigh, N.C.). A peroxidase conjugated streptavidin kit (Dako Inc., Carpinteria, Calif.) was used to detect binding for both evaluations, and samples were reacted with 3,3′ diaminobenzidine (DAB) substrate for visualization. Methyl green staining was used to identify background nuclei following both immunocytochemical techniques.

All animal studies were performed with protocols approved by the University of Arizona IACUC and according to the National Institutes of Health Guidelines for the Care and Use of Laboratory Animals (#85-23 Rev. 1985).

### EXAMPLE 1

HBPR/protein-modified (HBPR COLI/LM5, etc) coronary stents (3×8 mm) were evaluated for healing responses in the iliac arteries of New Zealand white rabbits. The coatings were formed on either 3×8 mm cobalt chromium bare metal stents or 3×8 mm cobalt chromium metal stents having a silane/Parylene™ base coat and a drug-eluting pBMA/pEVA/paclitaxel coat. The coatings are summarized in Table 1 below. Prior to protein or photo-protein coating, but following any silane/Parylene™ or pBMA/pEVA/paclitaxel coating the stents were EtO sterilized. Aseptic techniques were then used to apply the protein or photo-protein coatings.

For some stent samples, coatings were formed using photogroup-derivatized matrix proteins (photo-collagen and photo-laminin). Photogroup-derivatized matrix proteins were prepared as described in U.S. Pat. No. 5,744,515 (Clapper). A stent was placed into a 10×75 mm glass test tube (1 stent per test tube) and 1 mL of photo-collagen-I at a concentration of 200 ug/mL in 12 mM HCl was added to the tube. For photo-laminin-1 coatings, 1 mL of photo-laminin-1 at a concentration of 10 μg/mL in 0.1 M CBC buffer (0.1M sodium carbonate, 0.1M sodium bicarbonate) pH 9.0, was added to the tube. The stents were then shaken for 1 hr at 4° C. with mild agitation on a shaker. The stents were then illuminated using Dymax™ Bluewave 200 (Torrington, Conn.) with a 324 nm filter lens (59458; Oriel Corporation, Stratford, Conn.) for 2×30 sec (illuminated, rotated test tube 180°, and illuminated again). Following irradiation the stent was held with sterile Teflon coated forceps and gently agitated stent in sterile Endosafe™ water (Charles River, Charleston, S.C.). The stent was then blot dried on sterile Alpha™-10 wipes (Cole Parmer) and stored stents in sterile 96-well plate at 4° C.

For some stent samples, a silane layer was formed by immersing the (bare metal) cleansed cobalt chromium stents in a solution of 0.5% (w/v) γ-methacryloxypropyltrimethyl-silane in a mixture of IPA/water at room temperature for approximately 1 hour with shaking on an orbital shaker. After silane treatment, the stents were briefly rinsed in isopropyl alcohol and then baked in an oven at a temperature of 100° C. for approximately 1 hour.

For some stent samples, a Parylene™ layer was formed by placing the silane-coated stents in a Parylene™ coating reactor (PDS 2010 LABCOTER™ 2, Specialty Coating Systems, Indianapolis, Ind.) and then coating with Parylene™ C (Specialty Coating Systems, Indianapolis, Ind.) by following the operating instructions for the LABCOTER™ system. The resulting Parylene™ C coating was approximately 1-2 μm thickness.

For stents having a pBMA/pEVA/paclitaxel coating, a spray coating procedure was employed as follows. Spray coating was carried out using coating system such as that described in U.S. Publication No. 2004/0062875-A1. Coating was applied to the stent at a rate of 0.1 mL/min with a spray pressure of 1.3 PSI. The spray nozzle utilized was an ultrasonic nozzle operated at a power of 0.6 W (Sonotek). The spray head passed over the stent 40 times (described as the number of “passes”; 2 passes equals 1 cycle), as indicated. The total number of passes was selected to provide a final total coating weight of 30 μg/stent, and a final paclitaxel weight of 10 μg. The spray coatings were applied at a relative humidity of 30%.

For preparing the pBMA/pEVA/paclitaxel layer, a mixture of pEVA (33 weight percent vinyl acetate; Aldrich Chemical, Milwaukee, Wis.) at a concentration of 8 mg/ml; pBMA (337,000 average molecular weight; Aldrich Chemical, Milwaukee, Wis.) at a concentration of 20 mg/ml; and paclitaxel (Mayne Pharma, Paramus, N.J.) at a concentration of 12 mg/ml, was prepared in THF.

HBPR was prepared at a concentration of 10 mg/ml in 50% isopropanol/50% water solution. The HBPR was applied to the stents using the following spray parameters: 1.3 psi spray pressure, humidity <16%, 0.1 mL/min flow, 0.6 Watts Ultrasonic energy, 100 cycles, which resulted in 20-25 micrograms of HBPR deposited on the stents. Following HBPR coatings, the stents were placed in a box with nitrogen stream (low psi—<3) for 10 minutes. Following this, the stents were treated with UV radiation using a Dymax™ Bluewave 200 (Torrington, Conn.) with a 324 nm filter lens for 60 seconds with rotation.

HBPR-coated stents were placed into sterile microcentrifuge tubes (1 stent per tube). The following protein solutions were prepared in 0.1 M CBC buffer, pH 9.0:20 μg/ml laminin-5; 100 μg/ml laminin-1; and 20 μg/ml laminin-5 and 10 μg/ml collagen-I. The protein solutions in an amount of 70 μL were then individually dispensed into the test tubes causing bonding of the proteins to the HBPR component. The tubes were incubated at 4° C. overnight on shaker with mild agitation. The stents were then held with sterile Teflon coated forceps and gently agitated in Endosafe™ water. The stent was then blot dried on sterile Alpha™-10 wipes and stored stents in sterile 96-well plate at 4° C.

Table 1 summarizes the coatings prepared on the stents.

TABLE 1

| | Coating | | | | |
|---|---|---|---|---|---|
| Stent | Silane/ | pBMA/pEVA/ | | | |
| sample | Parylene ™ | paclitaxel | HBPR | Collagen type | Laminin type |
| 1 | + | + | − | — | Photo-Laminin-1 |
| 2 | + | + | + | — | Laminin-1 |
| 3 - control | + | + | − | — | — |
| 4 | + | − | − | — | Photo-Laminin-1 |
| 5 | + | − | − | Photo-collagen-I | — |
| 6 | + | − | + | — | Laminin-1 |
| 7 | + | − | + | — | Laminin-5 |
| 8 | + | − | + | Collagen-I | Laminin-5 |
| 9 - control | − | − | − | — | — |

Stent crimping onto 3×12 mm balloon catheters (EtO sterilized, RX Vision-E P/N SA2036149-302, Lot 6022352) was performed in a laminar sterile flow hood using a bioassay dish as sterile field. A stent crimper available from Machine Solutions, Inc. (Flagstaff, Ariz.) was disinfected with 70% ethanol was used to perform the process. One individual handled the catheter and positioned the stent and second individual used sterile forceps to pick up stents, crimp stents, open packages, and seal finished devices in sterile packages. Stents were first slightly crimped, the position of the stent adjusted if needed, then crimped a final time. All packaged devices were stored at 4° C.

The stents were then deployed into New Zealand white rabbits. A balloon inflation injury was performed to iliac arteries to denude the vessel of endothelium prior to stenting. Stents were deployed in both iliac arteries. The stents were explanted at 7 and 14 days (28 and 90 day explantations are also evaluated) and evaluated by light and scanning electron microscopy. On one stent half, BBI (bisbenzimide; Hoechst 33258) nuclei staining was performed. The remaining half of each stent was processed for scanning electron microscopy.

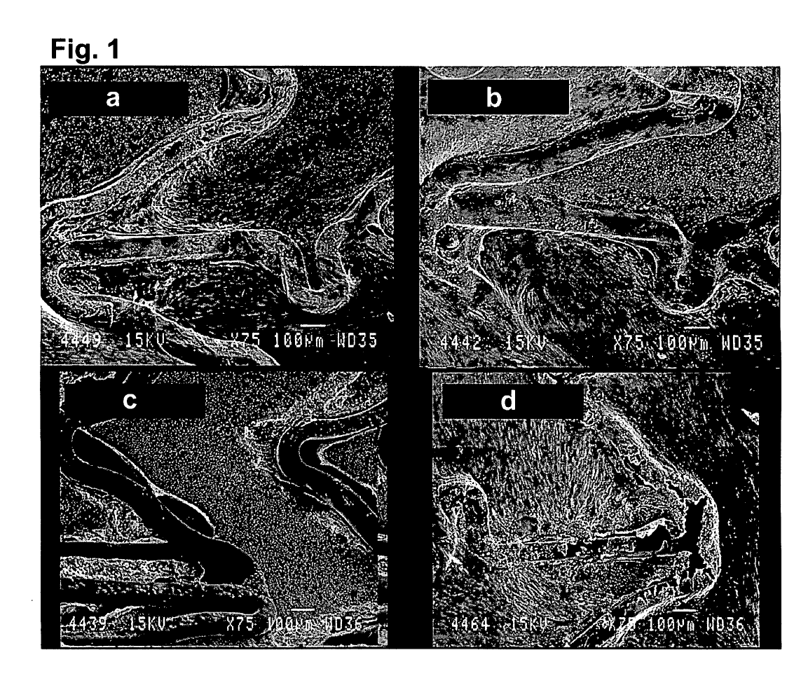

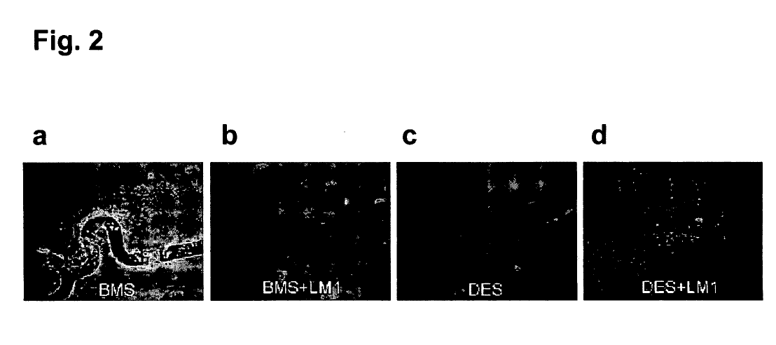

Results of the analysis of the control and coated stents explanted at day 7 show generally show improved endothelialization of the adhesion factor-coated stents. (See FIGS. 1 and 2) Stents having drug-eluting coating (DES) without any matrix protein coating were viewed as having the poorest endothelialization and also showed some fibrin deposits. Coatings formed from HBPR with an adhesion factor showed the best improvement in endothelialization for stents including the drug-eluting coating. Coatings formed from HBPR with an adhesion factor also generally supported very good endothelialization on stents without drug-eluting coating. Adhesion factor-based coatings showed no observable fibrin deposits.

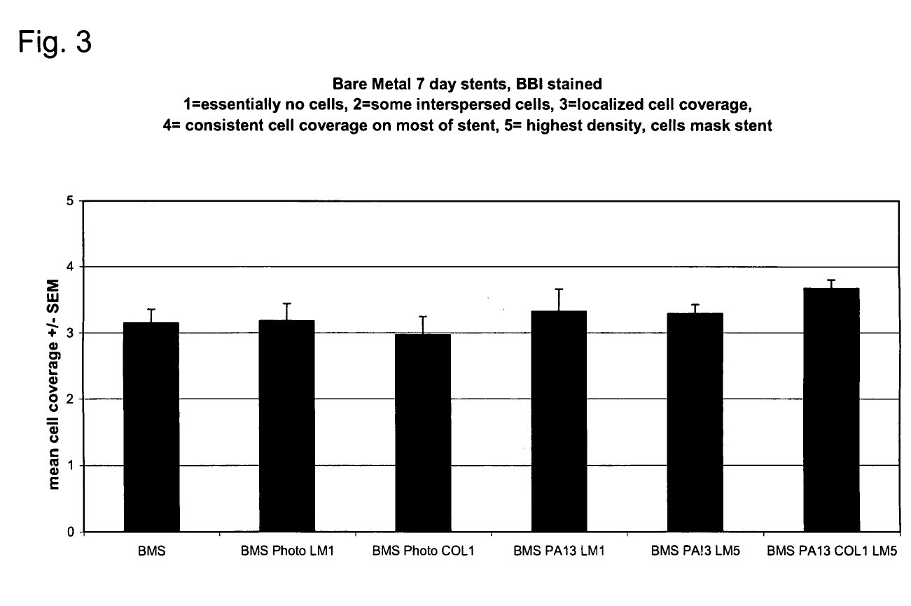

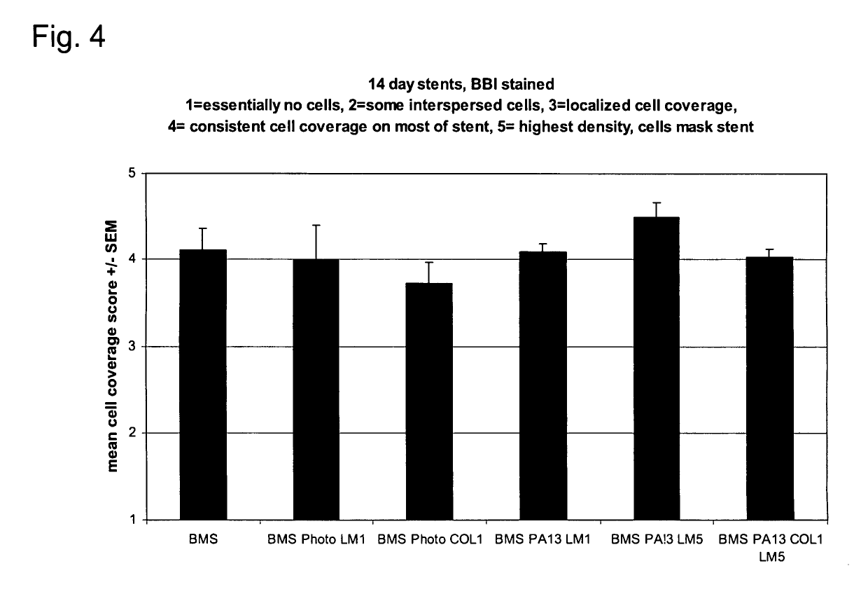

At time points (e.g., 7 and 14 day explantations), observations were made to determine endothelialization (See FIGS. 3 and 4), inflammation, and intimal fibrin content.

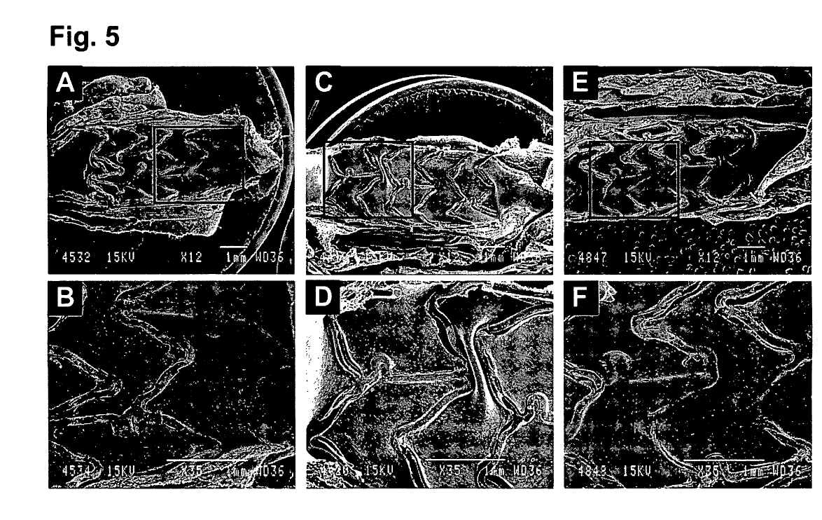

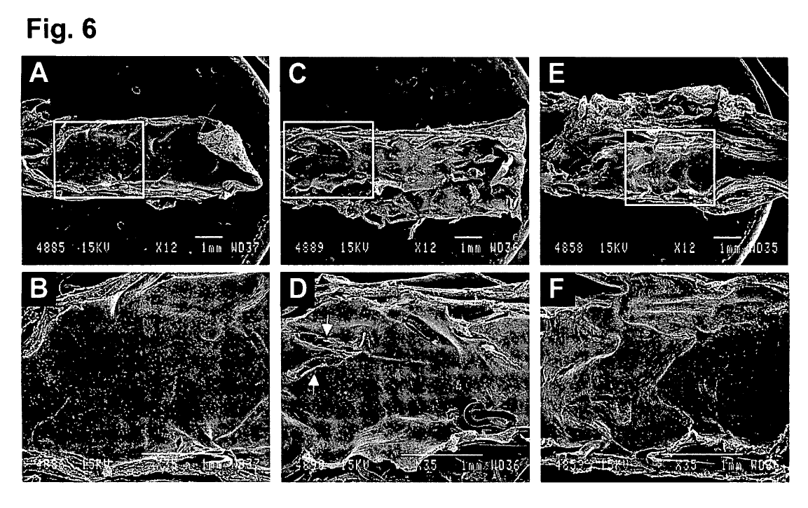

Observations also include assessments of percent luminal stenosis and neointimal thickness. FIGS. 5 and 6 show SEM images at 7 and 14 day time points, respectively, with sample stent 6 (BMS), stent 2 (BMS/Parylene/photo-collagen I), and stent 3 (BMS/Parylene/HBPR/laminin 1).

At seven days all stents showed the beginning of a pro-healing response, as observed the presence of a sub-monolayer levels of endothelialization.

However, at 14 days differences were seen in the endothelial response. While the surface of the BMS was trending towards endothelial cell hypertrophy, as observed by higher levels of endothelialization (endothelial cells attaching in an amount greater than a cellular monolayer), endothelialization of the collagen coated sample (i.e., stent 2) was modulated, tending towards the formation of a endothelial cell monolayer.

### EXAMPLE 2

Collagen 1 coatings, including those formed from photo-collagen, were prepared in non-fibrillar and fibrillar forms.

A photo-collagen-I solution for formation of a fibrillar coating was prepared. An aqueous solution of 12 mM HCl was cooled on ice and photo-collagen-I was added to provide a concentration of 3 mg/mL, and the solution kept on ice. The photo-collagen solution was diluted 1:3 with cold 12 mM HCl resulting in a concentration of 1 mg/ml.

The photo-collagen-I solution in an amount of 1.5 mL was centrifuged for 5 min at 14,000 RPM. Supernatant in an amount of 600 uL was transferred to a new tubes. A collagen-I (non-photo, lyophilized material) solution was prepared in chilled 12 mM HCl at 1 mg/mL and subjected to the same centrifugation and supernatant removal.

To 600 uL of the collagen-I and photo-collagen-I solutions were added 600 uL 0.1 M carbonate/bicarbonate buffer (CBC), pH 9.0, resulting in a pH>9.0. A collagen-1 solution was also prepared at pH 7.4 using phosphate buffered saline. After CBC addition the solutions were placed in a 37° C. orbital incubator, shaking at 200 RPM for overnight (˜18 hrs). The solutions were centrifuged at 2000 RPM for 15 min at room temperature. Supernatant was removed, leaving about 1 mL in the test tubes. Individual solutions were mixed briefly by vortexing, and 20 uL was dispensed onto a silane-treated glass slide and allowed to dry. The slides were rinsed with DI water, dried, and atomic force microscopy (AFM) analysis was performed.

No fibrils were observed with collagen-I at pH 7.4. Fibrils were observed at pH 9.0 for both collagen-I and photo-collagen-I. The photo-collagen-I fibrils were shorter and narrower than the collagen-I fibrils.

A photo-collagen coated parylene-coated steel tube was also prepared. Photo-collagen-I was prepared at 200 ug/mL in 12 mM HCl (pH 2.0). A parylene coated stainless steel tube was incubated in the photo-collagen-I for 1 hr at 4° C., shaking at 200 RPM. The tube was illuminated for 3 minutes, rinsed in DI water, and dried. No fibrils were observed by AFM.

### EXAMPLE 3

The thrombotic effect of the bare metal surfaces, photo-collagen coated surfaces and regular collagen surfaces was examined in a porcine ex-vivo AV shunt model, similar to the procedure as described in Hanson S. R., et al., (1980) “In vivo evaluation of artificial surfaces using a nonhuman primate model of arterial thrombosis,” J Lab Clin Med 95, 289-304.

For the photo-collagen coating, a Parylene-coated stent was soaked in photo-collagen-I (200 ug/ml, 12 mM HCl) for 1 hr at 4 C while shaking followed by an in-solution illumination for 3 min. The stent was then rinsed in water and dried.

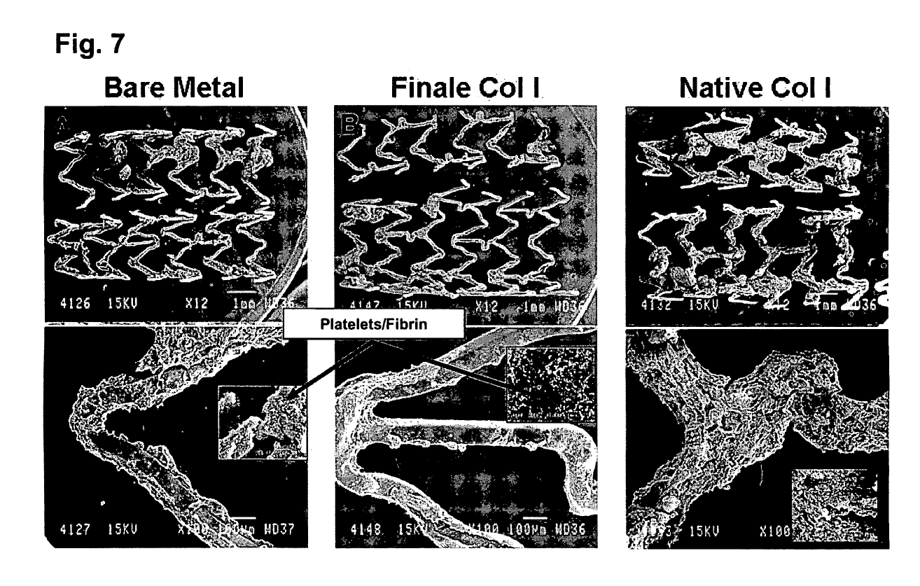

Results of the ex-vivo study are shown in FIGS. 7a-7c, showing excessive thrombosis on the collagen-coated stents (7c), and moderate, acceptable level of thrombosis on the photo-collagen coated stents (7b).

### EXAMPLE 4

In order to assess coating uniformity and defects, photo-collagen coated stents were stained with colloidal gold and visualized by microscopy. Stents were prepared with a Parylene coating as described herein. A stent was soaked in photo-collagen-I (200 ug/ml, 12 mM HCl) for 1 hr at 4 C while shaking followed by an in-solution illumination for 3 min. The stent was then rinsed in water and dried.

Stents were incubated 10 minutes in 30 nm colloidal gold as provided by the manufacturer (BBI International, GC30), for 5 min at room temp, then rinsed 3× in PBS. Air dried stents were observed with a Chroma filter 31000 on a Leica fluorescent microscope at 200× magnification.

### EXAMPLE 5

Various coated stents were prepared, having coatings were formed using photogroup-derivatized matrix proteins (photo-collagen and photo-laminin). The components present in the coatings are summarized in Table 2 below. The particular arrangements of the coating components in the coatings are described after the table.

TABLE 2

| | Coating | | | | | | | |
|---|---|---|---|---|---|---|---|---|
| Stent | | pBMA/ | | | | | | |
| sample | Parylene | pEVA/ | HBPR | 20GACL80LA | MD-HEX | IgG | Phot-Col | Phot-Lam |
| 10 | − | − | − | + | − | − | + | − |
| 11 | − | − | − | − | + | − | + | − |
| 12 | + | − | − | + | − | − | + | − |
| 13 | + | − | − | + | − | − | + | − |
| 14 | + | − | − | − | − | − | + | + |
| 15 | + | + | − | − | − | − | + | + |
| 16a | + | − | + | − | − | + | + | − |
| 16b | + | − | + | − | − | + | + | − |

Stent 10. Stainless steel 5×15 stents (Laserage) were first spraycoated with a multiblock copolymer composed of 20 wt % glycolide-caprolactone copolymer and 80 wt % lactide polymer (20GACL80LA) using an ultrasonic spray system (Sonotek). The spraycoating solution was prepared at 40 mg/ml in chloroform. The stents were then soaked for 1 hour in a 200 μg/ml Photo-Collagen I solution in 12 mM HCl, illuminated in solution for 3 minutes in front of a Dymax UV floodlamp, and rinsed in water.

Stent 11. Stainless steel 5×15 Laserage stents were first spraycoated with a polymerized maltodextrin containing hexanoate groups at 50 mg/ml in THF (commonly assigned U.S. patent application Ser. No. 11/724,553, filed Mar. 15, 2007, Chudzik) using an ultrasonic spray system (Sonotek). The stents were then soaked for 1 hour in a 200 μg/ml Photo-Collagen I solution in 12 mM HCl, illuminated in solution for 3 minutes in front of a Dymax UV floodlamp, and rinsed in water.

Stent 12. Parylene-treated stainless steel 5×15 Laserage stents were soaked for 1 hour in a 200 μg/ml Photo-Collagen I solution in 12 mM HCl, illuminated in solution for 3 minutes in front of a Dymax UV floodlamp, and rinsed in water. The stents were then spraycoated with a multiblock copolymer composed of 20 wt % glycolide-caprolactone copolymer and 80 wt % lactide polymer coating using an ultrasonic spray system using the same conditions as above (Sonotek). Finally, the stents were soaked for 1 hour in a 200 μg/ml Photo-Collagen I solution in 12 mM HCl, illuminated in solution for 3 minutes in front of a Dymax UV floodlamp, and rinsed in water.

Stent 13. Parylene-treated stainless steel 5×15 Laserage stents were soaked for 1 hour in a 200 μg/ml Photo-Collagen I solution in 12 mM HCl, illuminated in solution for 3 minutes in front of a Dymax UV floodlamp, and rinsed in water. The stents were then spraycoated with a multiblock copolymer composed of 20 wt % glycolide-caprolactone copolymer and 80 wt % lactide polymer coating using an ultrasonic spray system (Sonotek).

Stent 14. Parylene-treated stainless steel 5×15 Laserage stents were soaked for 1 hour in a 200/10 μg/ml Photo-Collagen I/Photo-Laminin I solution in 0.1 M CBC, illuminated in solution for 3 minutes in front of a Dymax UV floodlamp, and rinsed in water.

Stent 15. Parylene-treated stainless steel 5×15 Laserage stents were first spraycoated with a 50/50 PBMA/PEVA coating using an ultrasonic spray system (Sonotek). The spraycoating solution was prepared at 40 mg/ml in chloroform. The stents were then soaked for 1 hour in a 200/10 μg/ml Photo-Collagen I/Photo-Laminin I solution in 0.1 M CBC, illuminated in solution for 3 minutes in front of a Dymax UV floodlamp, and rinsed in water.

Stent 16a. A conjugate of a heterobifunctional polymer (HBPR), mouse IgG, and Photo-Collagen I was prepared by mixing the components in 0.1 M CBC to obtain final concentrations of 2/0.175/0.35 mg/ml HBP/IgG/Photo-Collagen I. Parylene-treated stainless steel 5×15 Laserage stents were then coated with the conjugate by soaking stents in the conjugate solution overnight at 4° C., illuminating in solution for 3 minutes in front of a Dymax UV floodlamp, and rinsing in water.

Stent 16b. Also prepared HBP/Photo-Collagen I conjugate at 2/0.35 mg/ml in 0.1 M CBC and HBP/IgG conjugate at 2/0.175 mg/ml in 0.1 M CBC. Parylene-treated stainless steel 5×15 Laserage stents were then coated with each conjugate by soaking stents in the conjugate solution overnight at 4° C., illuminating in solution for 3 minutes in front of a Dymax UV floodlamp, and rinsing in water.

What is claimed is:

1. A coated intravascular medical device comprising:
a body member comprising a metal or metal alloy and having a body member surface, and
a coating on the body member surface comprising:
a first layer that is in contact with tissue or body fluid, wherein the first layer comprises, predominantly, an adhesion factor and a photogroup; and
a second layer located between the body member surface and the first layer, the second layer comprising a polymeric material, and wherein the photogroup bonds the adhesion factor to the polymeric material.

2. The coated intravascular medical device of claim 1 wherein the photogroup is pendent from the adhesion factor.

3. The coated intravascular medical device of claim 1 wherein the adhesion factor comprises collagen.

4. The coated intravascular medical device of claim 1 wherein the polymeric material comprises poly(para-xylylene).

5. The coated intravascular medical device of claim 1 having a body member in the form of a stent.

6. A coated intravascular medical device comprising:
a body member having a body member surface, and
a bioactive agent-releasing coating on the body member surface comprising:
a first layer that is in contact with tissue or body fluid, wherein the first layer comprises, predominantly, a adhesion factor and a photogroup; and
a second layer located between the body member surface and the first layer, the second layer comprising a polymeric material and a bioactive agent, and wherein the photogroup bonds the adhesion factor to the polymeric material.

7. The coated intravascular medical device of claim 6 wherein the polymeric material of the second layer comprises a hydrophobic polymer.

8. The coated intravascular medical device of claim 6 wherein the polymeric material of the second layer comprises a degradable polymer.

9. A method for improving the endothelialization of a surface of an implantable medical article, the method comprising steps of
providing a medical article with a coating comprising a polymeric material, a bioactive agent, an adhesion factor, and a photogroup that bonds the adhesion factor to the polymeric material; and
implanting the coated medical article in a subject, wherein the coating promotes a level of endothelialization in the subject that is greater than a level of endothelialization observed without the adhesion factor and photogroup.

10. A method for modulating the endothelialization of a surface of an implantable medical article, the method comprising steps of
obtaining information regarding the endothelialization of a surface of the medical article, wherein the medical article has a first level of endothelialization when implanted into a subject after a period of time, and the first level of endothelialization is associated with an undesirable tissue response;
providing a medical article with a coating comprising at least one adhesion factor and a photogroup to form a coated medical article; and
implanting the coated medical article in a subject, wherein the coating promotes a second level of endothelialization in the subject that is less than the first level of endothelialization after the period of time.

11. The method of claim 10 wherein the step of obtaining, the undesirable tissue response is smooth muscle cell hyperproliferation.

12. The method of claim 10 wherein the subject is a human and the period of time is about two weeks, or greater than two weeks.

13. The method of claim 10 wherein the period of time is about four weeks.

14. The method of claim 10 wherein the step of providing comprises providing a coating to the medical article that comprises an adhesion factor selected from collagens and laminins, active portions thereof, or binding members thereof.

15. The method of claim 14 wherein the step of providing comprises providing a coating to the medical article that comprises collagen I, active portions thereof, or binding members thereof.

16. The method of claim 10 wherein the step of providing comprises providing a coating to an intraluminal prosthesis.

17. The method of claim 14 wherein the step of providing comprises providing a coating to an intravascular prosthesis.

18. The method of claim 10 wherein the step of implanting is performed by delivering the medical article to an intravascular location in the subject and maintaining for a period of time sufficient to cause the formation of an endothelial layer of cells on a surface of the medical article.

19. A coated intravascular medical device comprising a body member comprising a bioresorbable material, and a coating on the bioresorbable body member, the coating comprising adhesion factor and a photogroup, wherein the photogroup allows the adhesion factor to form a coated layer on the surface of the body member.

20. The coated intravascular medical device of claim 19 wherein bioresorbable material comprises a biodegradable polymer and the photogroups bond the adhesion factor to the biodegradable polymer.

21. The coated intravascular medical device of claim 19 wherein photogroups bond adhesion factor together.
